(19) United States
(12) Patent Application Publication
(10) Pub. No.: US 2025/0111922 A1
(43) Pub. Date: Apr. 3, 2025

(54) METHOD AND SYSTEM FOR THE TREATMENT OF PROGRESSING MYOPIA

(71) Applicant: Hakan Kaymak, Düsseldorf (DE)

(72) Inventors: Hakan Kaymak, Düsseldorf (DE); Hartmut Schwahn, Tübingen (DE)

(21) Appl. No.: 18/833,129

(22) PCT Filed: Jan. 26, 2022
(86) PCT No.: PCT/EP2022/051750

§ 371 Date: Jul. 25, 2024

(51) Int. Cl. G16H 20/30; A61B 3/10; A61B 5/00; G16H 10/60

(52) U.S. Cl. CPC G16H 20/30; A61B 3/10; A61B 5/4848; G16H 10/60

(57) ABSTRACT

The present invention concerns the field of ophthalmology and pediatric eye care and prevention and provides methods and systems for the treatment of myopic subjects and for myopia management, in particular, a computer-implemented method for the automatic evaluation and monitoring the efficiency of a myopia treatment and a computer-based system having implemented the same.

### TECHNICAL FIELD

The present invention concerns the field of ophthalmology and pediatric eye care and prevention and provides methods and systems for treating progressing myopia and for myopia control management.

### BACKGROUND

Myopia, also known as short-sightedness or near-sightedness, is a condition of the eye where light from a faraway object does not focus on the sensory retina, but instead in front of the retina. This causes distant objects to appear blurry while close objects can be seen clearly. In optical terms, myopia is characterized in an anatomical mismatch between axial length of the eye ball and the optical power of the refractive elements of the eye, in particular the cornea and eye lens (refractive error). While refractive correction by corrective lenses or glasses may, in most cases, compensate for an eye's refractive error and restore clear vision of distant objects, the eye's anatomical mismatch prevails.

On contrast, emmetropia is the normal or “healthy” state of a subject's eye where a faraway object (at infinity distance) is in sharp focus on the eye's retina with the eye's accommodative apparatus in the anterior segment of the eye, comprising lens, zonula fibres and ciliary muscles, being in a neutral state. This is achieved when the refractive power of the cornea and eye lens and the axial length of the eye (in other words: the effective distance between the eye's refractive elements in the anterior segment and the sensory retina in the posterior segment of the eye) balance out. A condition where the eye's refractive elements focuses the rays of the faraway object exactly on the retina, resulting in perfect vision of distance objects. A subject's eye in the state of emmetropia requires no corrective lenses. The development of an eye towards emmetropia is known as emmetropization. During the process of emmetropization in infancy, the axial length increases in line with the focal length of the eye's optics until it reaches the “adult” axial length, according to literature (Sorsby A, Leary G A. “A longitudinal study of refraction and its components during growth.” Spec Rep Ser Med Res Counc. 1969; 309:1-41) at the age of 13 years and is thought to remain stable thereafter. Newborns are typically hypermetropic and then undergo a “myopic shift” to become emmetropic. Emmetropization is an active and process during the eyes' development from child to adulthood, which, due to the high accuracy of the balancing between the eye's refractive power and axial length required for good vision, is considered to be actively controlled by one or more feedback loops.

Now, in the event that the axial growth rate is higher than normal, i.e. in excess of what is required to achieve or maintain emmetropia during juvenile eye growth, axial myopia develops. If the myopic eye then continues to grow in axial direction at an excess rate, the degree of myopia increases and eventually high myopia (−5 Dpt. or less; WHO definition 2015) may develop. Both, genetic and environmental factors, influence axial growth and intervene with the normal process of emmetropization and thus the growth (rate) of the child's eyes.

Myopia the most common eye disorder in developed countries. There is a high prevalence of myopia of 80% to 90% in young educated adults in East Asia and of about 50% in western countries. Myopia prevalence increases with age, is generally associated with less time spent outside, is more prevalent in certain ethnic populations, and also has a genetic association. Myopia is considered an eyesight-threatening disease, and in regions of high prevalence myopia has become the leading cause of blindness. Axial myopia induces various specific complications, including, but not limited to cataract formation, retinal detachment from (peripheral) retinal tears, myopic foveoschisis, macular hole and retinal detachment, peripapillary deformation, posterior staphyloma, dome-shaped macula, choroidal/scleral thinning, myopic choroidal neovascularization, and glaucoma (primary open angle glaucoma). The risk level for these conditions increases with the myopia grade and consistently with the degree of excessive axial elongation. The myopic population increases globally and the increase in severity of the impact of myopia on global health and, of course, the health systems is easily predictable.

Of note, the present invention mainly concerns axial myopia, as characterized by an excessive axial elongation. There are other forms of myopia and conditions where myopic refractive error of the eye can occur such as refractive myopia as attributed to the condition of the refractive elements of the eye, including and in particular curvature myopia which is attributed to an excessive, or increased, curvature of one or more of the refractive surfaces of the eye, especially the cornea, including the condition of Keratoconus, and myopia resulting from high corneal and lenticular power (Cohen syndrome); and index myopia which is attributed to a variation in the index of refraction of one or more of the ocular media. are not primarily encompassed by the following considerations but may well exist in addition to axial myopia and in that respect would not leave the scope of the present considerations on axial myopia and myopia management. These forms of myopia are not primarily encompassed by the following considerations but may well exist in addition to axial myopia and in that respect would not leave the scope of the present considerations on axial myopia. These forms of myopia also are often congenital and forms of infantile myopia which are present at birth and persist through infancy.

On contrast, most forms of axial myopia are acquired during development. A particular form of interest in the present consideration is “school myopia” which appears during childhood, particularly in the school-age years. In a very typical scenario, children upon entrance in primary school or, in a more particular scenario, upon entering secondary school, develop myopia due to an increase in axial growth, possibly triggered by a change in the visual environment of the child; this myopic shift may also occur in young adults upon entering university or comparable education or work conditions. This incidence of myopia or myopic shift is caused by a (sudden) increase in axial growth (myopic growth) which is in excess of the eye's normal growth which otherwise would lead to or maintain emmetropic state (emmetropic growth).

The last five years saw a very significant rise in clinical applications of new methods and tools for the treatment and prevention of myopia progression and/or for delaying the incidence of (school) myopia in children and adolescents. Various treatments, such as topical low-dose atropine, novel spectacle lens designs, multifocal contact lenses, and orthokeratology have been proven in randomized controlled trials (RCT) to slow down axial elongation in children. Concomitantly, a new discipline among

Optometrists and Ophthalmologists has developed: “myopia management”, also known as “myopia control” which generally concerns the proactive and systematic implementation of one or more strategies to prevent or slow down the progression of myopia in children. In evidence of effective means for myopia control, several authoritative bodies have recently declared that the new standard of care for myopia must include early detection of children at risk and managing myopia progression with evidence-based treatments either directly or via referral or co-management (Modjtahedi et al. “Reducing the Global Burden of Myopia by Delaying the Onset of Myopia and Reducing Myopic Progression in Children: The Academy's Task Force on Myopia.” Ophthalmology. December 2020; doi: 10.1016/j.ophtha.2020.10.040). The overall goal of myopia management is to stop the progression of both axial length growth and refractive error; a general goal is to prevent that a subject's axial length remains less than 26.0 mm and the refractive error, as expressed by spherical equivalent refraction (SER) remains above −6.0 D.

However, in the individual subject, myopia management can be challenging: Currently, 161 independent loci for refractive error, which explain 8% of the variance of refraction (SER) in adults and can discriminate myopia from hyperopia with accuracy of 0.77, have been identified (Tedja M S et al. “Genome-wide association meta-analysis highlights light-induced signaling as a driver for refractive error.” Nat Genet. 2018; 50 (6): 834-48). In addition, there are well established environmental myopia risk factors, including, but not limited to extended near work and minimal outdoor exposure (Tideman J W L, et al. “Environmental factors explain socioeconomic prevalence diferences in myopia in 6-year-old children.” Br J Ophthalmol. 2017. doi: 10.1136/bjophthalmol-2017-31029). Accordingly, a subject's individual risk to develop myopia or even high myopia depends on many genetically determined (nature) and environmental (nurture) factors, which each vary to a great extend in each individual. This individual variability in the “myopia driving” factors may also cause the observed variance in a subject's susceptibility to a particular myopia therapy option, i.e. the therapeutic efficiency of the myopia therapy.

Currently, there is a variety of options for the treatment of myopia in children and adolescents available which have been proven to be effective in reducing excessive axial growth in subjects diagnosed of current progressive or incident axial myopia. However, not every treatment option is as efficient in all subjects. Without wishing to be bound to the theory, the phenomena of “non-responder” or “slow-responder” or “low responder” to a particular therapy option, is related to genetic, physical, physiological, psychological, environmental or social factors or combinations thereof. But while one particular therapy may not be efficient in one subject, an alternative therapy option or a combination of two or more therapy options may well be.

In the myopia management, clinicians thus would need to closely monitor the therapeutic efficiency of a myopia therapy option selected for a patient and to decide whether the proper myopia therapy option had been selected in the first place or if a change in the therapeutic strategy is indicated. Such change in therapy may comprise to substitute the current therapy option for another or alternative treatment option or to intensify or reduce the current therapy option or to combine two or more alternative treatment options for greater efficiency. Some therapy options are considered more efficient than others, sometimes for the costs of unwanted side effects or tolerability. Myopia treatment commonly needs to be pursued for several years and possibly until adulthood. It is also of interest for the clinician to identify the particular point of time when the treatment goal is about to be reached and the treatment may be tapered out or ended. All known options of myopia treatment to date require high treatment adherence and compliance to be efficient, but since many treatment options are laborious and require daily attention and are thus cumbersome to follow, especially for young children that need to be supervised by their parents and for pubescent teens that do not want to be supervised by their parents.

It is thus desired to select and provide to the children, parents and adolescents an individualized and optimal therapy in myopia control management and to identify an optimal treatment regimen as early as feasible, as a less effective therapy option would waste valuable time to efficiently stop or reduce the excessive eye growth towards myopia or even high myopia, which is known to be irreversible.

There are theoretical approaches known to date which employ calculations of trajectories and prospective outcomes of the myopia progression of a subject. These known calculations and formulae require the assessment of many biometric parameters such as axial length and refractive error. In US 2017/0209036 A1 is disclosed a method for predicting a future axial elongation of the eye as a function of the prior refractive change, current axial length and age, in particular by estimating the percentile of a spherical equivalent refraction and an expected trajectory of refractive development. However, such methods fall short of providing to the treating eye doctor or eye care professional a clear indication whether or not the current myopia treatment option is effective enough to efficiently treat progressing myopia. in practical use. The method also requires the calculation of trajectories and comparisons which calculations are error prone if not employed properly or the parameters are not assessed at the required accuracy and repeatability. Such known approaches all require the assessment of a subject's refractive error (e.g. as expressed in spherical equivalent SER) which at one hand is tedious to assess in children and requires competent and educated personnel and special equipment and still show lack of accuracy and repeatability, and on the other hand is not directly connected to the subject's axial growth as refractive error has many components and causes and is mainly dictated by the temporary conditions refractive elements of the anterior segment of the eye but not the posterior pole where excessive eye growth and permanent increase in axial length actually occurs.

Other current attempts to monitor myopia therapy rely on the comparison of a patient's actual course of axial length development and compare the course with some normative data which is derived from cross-sectional epidemiologic studies. Due to the epidemiologic nature of such normative data, the comparison of an individual's current axial length only returns a statement about the general risk of individuals having the same axial length to develop myopia or high myopia. But given the fact that the subject currently treated is already diagnosed with progressive myopia, such information is of no practical use and relevance, and in particular such comparison does not provide any reliable information on the actual efficiency of the current myopia treatment option and provides no guidance for the treating eye doctor or eye care specialist on how to manage the subject's individual myopia therapy (myopia control management).

It therefore is an object present invention to provide methods and means for myopia control management, in particular methods and means to reliably monitor the therapeutic efficiency of any myopia therapy option and to provide a simple and therapeutically relevant indication for the clinician on the current status and efficiency of a current myopia treatment.

### SUMMARY OF THE INVENTION

The invention particularly pertains to the following embodiments and combinations thereof:

A first and primary embodiment of the present invention is a computer-implemented or implementable method for automatic evaluation and monitoring or controlling of the efficiency of a myopia treatment of a subject's eye, in particular of an eye which is diagnosed of current or incident axial myopia, more particular progressive myopia, specifically school-myopia, in children and adolescents. The method of the invention comprises the steps of:
(a) obtaining the current age (CA) of the subject;
(b) obtaining the current axial growth rate (AG) of the subject's eye;
(c) calculating a maximum tolerable axial growth rate (TAG) as a function of the subject's current age (CA);
(d) comparing the AG with TAG; and
(e) generating an output on the basis of said comparison, selected from:
   (i) output comprising the information of currently insufficient or ineffective myopia therapy for AG greater than TAG, and
   (ii) output comprising the information of currently adequate or effective myopia therapy for AG smaller than TAG or equal (TAG criterion).

In a particular variant of this embodiment, the method consists or essentially consist of steps (a) to (e) and does not require any additional substantial or even essential preceding or in between steps, such as the assessment or the eye's refractive state, in particular by means of subjective refraction and/the retrieval of data and parameters on the current and previous refractive state from a database or patient's record.

In a 2nd embodiment, the TAG function is particularly related or corresponds to the respective average axial growth data obtained from a cohort of children, who do not develop axial myopia during childhood and adolescence, i.e. an emmetropic cohort.

A 3rd embodiment is a particular embodiment of the first or the second embodiment, wherein TAG is further a function of the subject's sex(S). In a particular embodiment thereof, the TAG function is related or corresponds to the respective average axial growth data obtained from a cohort of children of corresponding sex(S), who do not develop axial myopia during childhood and adolescence, i.e. a sex matched emmetropic cohort.

A 4th embodiment is a particular embodiment of the first or the second or the third embodiment, wherein TAG is further a function of the subject's ethnicity (E). In a particular embodiment thereof, the TAG function is related or corresponds to the respective average axial growth data obtained from a cohort of children of corresponding ethnicity (E), who do not develop axial myopia during childhood and adolescence; i.e. an ethnicity matched emmetropic cohort.

A 5th embodiment is a particular embodiment of the first or the second or the third embodiment, wherein TAG [mm/yr] is computed as a combination of a linear equation (y=b+ax) and a constant (y=c), which follows the following equation system:

$$TAG [ mm / yr ] = b [ mm / yr ] - a [ mm / {yr}^{2} ] \times CA [ yr ] , and \qquad ( eq . 1 )$$

$$TAG [ mm / yr ] \geq c [ mm / yr ] \qquad ( eq . 2 )$$

That is, TAG follows a linear function based on parameters a, b dependent on CA, but becomes a constant c (eq. 2) for all values of TAG would become smaller than c according to the linear equation (eq. 1).

In embodiment 6 c is from 0.08 to 0.12; in embodiment 7 c is 0.10, which is a preferred embodiment.

Embodiment 8 is a variant of any one of the aforementioned embodiments, where b is from 0.38 to 0.42 and a is from −0.022 to −0.024.6. Embodiment 9 is a variant of any one of the aforementioned embodiments, where bis 0.395, if the subject's ethnicity (E) is “caucasian”, and b is 0.420 if E is “east asian”. Embodiment 10 is a variant of any one of the aforementioned embodiments, where a is −0.022, if the subject's sex(S) is “male”, and a is −0.024, if S is “female”.

In embodiment 11, which is a variant of any one of the aforementioned embodiments, step (b) comprises the steps of: (b1) retrieving a past axial length (PAX) [mm] from that eye from a previous point of time and the previous age (PA) [yr] of the subject at that previous point of time from a database record; (b2) obtaining the current axial length (CAX) from that eye; and (b3) computing AG according to formula: AG=(CAX-PAX)/(CA-PA)

In a 12th embodiment, which is a variant of any one of the aforementioned embodiments, the subject's current age (CA) is between 6.00 and 16.50 yr.

In a 13th embodiment, which is a variant of any one of the aforementioned embodiments, step (a) comprises the steps of: (a1) obtaining the subject's date of birth; (a2) obtaining the current date, which is the date where the measurement of subject's CAX takes place; and (a3) computing the CA from the difference between said dates to retrieve subject's CA in a years [yr]+ fraction of years, preferably at an accuracy of 1 month or at an accuracy of 10/12 or 0.083 yr.

In a 14th embodiment, which is a variant of any one of the aforementioned embodiments, the subject's eye's AG is assessed at an interval of 6 months (0.50 yr) to less than 12 months (1.00 yr) to monitor the efficiency of the myopia treatment of the subject's eye

A 15th embodiment of the invention is a computer-based system for automated evaluation and monitoring of myopia treatment efficiency, comprising in a computing device (23) a computer memory (26) for storing program instructions and a CPU (25) for operating the stored program instructions, said program instructions comprising the steps (a) to (e) of the method according to any one of the aforementioned embodiments 1 to 14.

In a 16th embodiment, the computer-based system is further comprising a database (24), in data communication with that CPU (25) or memory (26), for storing current axial length (CAX) of a subject's eye in connection with the subject's current age (CA) and for retrieving one or more past axial lengths (PAX) and the subject's one or more previous ages (PA) connected thereto.

In a 17th embodiment, the computer-based system is further comprising a biometry device (21) for assessing a current axial length (CAX) of a subject's eye. The computer-based system may further a data interface (22) in data communication with the biometry device (21) and the CPU (25) and/or memory (26)

A 18th embodiment is a computer program product comprising a non-volatile readable storage medium or data server having embodied program instructions, said program instructions comprising the steps (a) to (e) of the method according to any one of aforementioned embodiments 1 to 14.

A 19th embodiment of the invention is a method for treating myopia in a subject, the method comprising tracking or monitoring the efficiency of the myopia treatment by the method comprising the steps (a) to (e) according to the embodiment described herein.

In a 20th embodiment, the method of treating myopia according to embodiment 19 further comprises the step of:
(f) if in step (e) the output comprises the information that the myopia treatment is currently insufficient or ineffective, a further treatment measure is taken, the further treatment measure being selected from: intensifying the current treatment, in particular by increasing the dosage of the current treatment option, choosing an alternative myopia treatment option, combining the current treatment option with at least one additional myopia treatment option, and taking measures to ensure or intensify subject's good adherence to therapy.

A 21st embodiment concerns particular variants, where the subject has or is at risk of developing myopia, in particular progressive myopia and/or high myopia; the myopia is primarily or essentially characterized in an excessive axial growth of the subject's eye (axial myopia). More particular, the myopia is a condition selected from: progressing myopia, school myopia, and late-onset myopia.

A 22nd embodiment of the invention is a method for preventing or treating a condition in a subject, the subject suffering from or being at risk of developing a condition which is a sight-threatening disease selected from: glaucoma, in particular open-angle glaucoma, cataract, in particular nuclear, cortical, and posterior subcapsular cataract, peripapillary deformation, posterior staphyloma, dome-shaped macula, choroidal/scleral thinning, myopic choroidal neovascularization retinal tears, retinal detachment, myopic maculopathy, and myopic macular degeneration, the method comprising tracking or monitoring the efficiency of the myopia treatment by the method comprising the steps (a) to (e) and optionally (f) according to the embodiments described herein. More particular the subject is suffering from myopia or high myopia or is at risk of developing high myopia.

In the methods according to the aforementioned embodiments the myopia treatment option is selected from: bifocal or multifocal contact lenses, orthokeratology lenses, multifocal spectacle lenses, pharmacological intervention, in particular, low-dose atropine, defocus incorporated multiple segment (D.I.M.S.) spectacle lenses and equivalent spectacle lens designs, regimen of increased outdoor activities (e.g. at least 2 hrs/day), high illumination daylight conditions, in particular light spectra corresponding to color temperatures of 5500 K (CIE D55 or F1, F5, or F7) or higher and at illumination levels of 10,000 lux or more, repeated red light stimulation, and any practical combinations thereof. In a more particular variant, the myopia treatment option is selected from: low-dose atropine treatment, in particular at doses of 0.01 to 0.05% Atropine, defocus incorporated multiple segment (D.I.M.S.) spectacle lenses and any practical combination thereof. In a more particular variant, the myopia treatment option is selected from various (increasing) doses of atropine treatment, in particular the myopia treatment option is selected from: 0.01%, 0.02%, 0.025%, and 0.05% atropine. In a more particular variant, the myopia treatment option is selected from these various (increasing) doses of atropine treatment, either in combination with defocus incorporated multiple segment (D.I.M.S.) spectacle lenses.

### BRIEF DESCRIPTION OF THE DRAWINGS

FIG. 1 shows modelled criteria curves “IAG” and “TAG” for the automated assessment of therapeutic efficiency of a myopia treatment option in the method according to the invention.

FIG. 2 schematically depicts a preferred embodiment of system for employing the method according to the invention for the automated assessment of therapeutic efficiency of a myopia treatment option

### DETAILED DESCRIPTION OF THE INVENTION

The present invention solves the underlying technical problem by providing a method of treatment and means for a method of treatment, said method and means are mainly characterized by an analysis of the current status of a subject's individual therapy to prevent or reduce myopia progression, the analysis is based, and in particular is exclusively based, on the subject's current axial length growth as assessed as axial growth rate, i.e. change of length per time, as expressed in particular as fraction of length [mm] per [yr] ([mm/yr]); with axial length measured with high accuracy automated biometry devices in [mm] at an accuracy of 0.001 mm and referred to subject's age in years at an accuracy of years [yr]+ fraction of years, preferably at an resolution of 1 month or an accuracy of 10/12 or 0.083 yr.

Advantageously, the method of the invention follows a practical approach which has been proven successful and highly reliable in routine myopia control management. In particular, it does never require obtaining a subject's (previous or actual) refractive state—as assessed in particular by known and established methods of subjective refraction or objective refraction, as these methods are cumbersome to perform with younger children and much often are error prone and, most relevantly, often lack good repeatability which, in turn, adversely affect longitudinal analyses of eye's myopia progression and practical evaluation and monitoring of the therapy.

The invention make use of the latest progress in the development and provision of devices to measure the axial length of a subject's eye by means of optical biometry: By these devices axial length can be easily and objectively assessed in an automated fashion and at high accuracy and repeatability in a short time. It goes without saying, good intra-individual repeatability is essential for useful assessment of a subject's axial growth rate. The method of the invention is adapted to the current devices' degree of accuracy and repeatability, and does not require higher accuracy than currently be achievable by these devices. By employing a simplistic and practical approach, the method of the invention is robust against some lack of accuracy and less error prone. Yet, the inventors surprisingly found that the method allows for a fast and very reliable evaluation and monitoring of the efficacy of a selected myopia therapy option, even after a short treatment or observation period of even less than 12 months. As the method of the invention is robust, with the advent of devices that measure axial length with higher accuracy and repeatability, a shorter observation period of 8 or 7 or 6 months or less is sufficient to determine and decide on the efficacy of a current myopia therapy option.

The method of the invention not only allows to monitor or track a current myopia treatment, it may also be used in general myopia management to assess whether or not myopia progression is so high to require a strong treatment or low to allow for a mild treatment or intervention, if any at all. It may also be employed in periodic myopia screening and diagnosis to identify subjects with progressing myopia, in particular in children at the transition from kindergarten to elementary school or from elementary to primary school or from primary to secondary school.

Every myopia therapy aims to reduce or prevent (further) progression of myopia. That is, the diagnosed refractive state of the eye remains stable and myopia does not progress, i.e. become more severe. Noteworthy, while a slight reduction in the degree of myopia, i.e. a hyperopic shift, might be observed in some therapies, in particular in the first phase of the therapy, as measured, e.g., by objective refraction (OR), due to adaptive processes in mainly in the eye's refractive apparatus in the anterior segment, there is no case described where excessive axial length which has caused the diagnosed (axial) myopia in the first place is reduced; there is no inverse axial growth or shrinking. Thus one can expect as the outcome of a causal myopia therapy a full halt in myopia progression at best, but never a reversal of progression and reduction of severity of the diagnosed myopia. Any negative medical consequences and risks associated with excessive eye growth and increased axial length cannot be avoided or ameliorated once the eye has assumed an increased axial length.

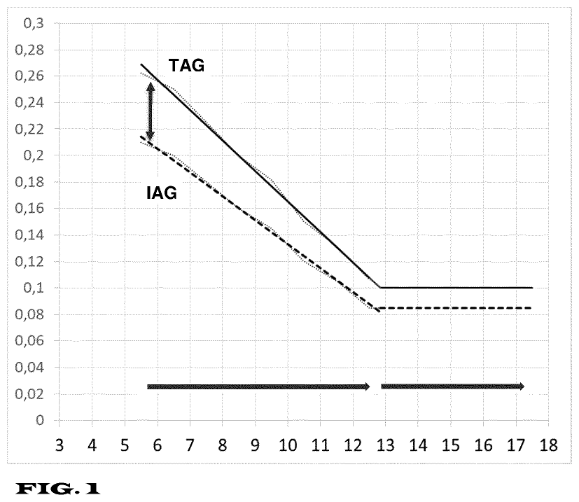

The model underlying the TAG criterion is particularly related or corresponds to the respective average axial growth data obtained from a cohort of children, who do not develop axial myopia during childhood and adolescence, i.e. an emmetropic cohort. Parameters on normal axial growth have been extracted from longitudinal and cross-sectional analyses of cohorts in the clinic of the inventor's over the last decade. This is also reflected by the IAG-curve (FIG. 1), which resembles the ideal situation for the growth rate of a myopic eye as it may be subject to myopia control management.

According to the invention, a subject's actual axial growth under myopia therapy is assessed and put into relation to the eye growth parameters of a matched cohort and under consideration of practical safety margins, taking into account the actual accuracy of the biometric devices for measurement of axial length and their repeatability in clinical routine, which results in a clear, safe and relevant indication for the eye doctor or eye care professional whether or not the current myopia therapy is sufficiently effective of requires further management and adaptation of the current therapeutic strategy. This is disclosed herein in particular as the TAG criterion (a preferred example is disclosed in FIG. 1).

In the context of the present invention and disclosure, a myopia therapy is considered “sufficient” or “effective”, if the expected overall myopia progression or “myopic shift” in a young subject diagnosed with current or incident axial myopia until reaching adulthood is not more than an additional −1.5 Dpt. This reflects about the “myopic shift” also gained in an emmetropic child following a normal growth pattern from a “physiological” hyperopia at young ages to a neutral refraction, i.e. emmetropia, at adulthood. The model and criterion underlying the present invention is based on the finding that any pathological excessive axial growth leading to progressive myopia is a superimposed the “normal” eye growth of an emmetropic child following a normal physiological growth pattern. It follows, that any myopia therapy that restores eye growth in children and adolescents diagnosed with progressive axial myopia to a level of “normal” eye growth as it would be the case in an emmetropic child following a normal and physiological growth pattern, is considered to provide 100% (i.e. full or maximum) therapeutic efficiency. Known assessments of therapeutic efficiency fall short in the provision of an objective measure as they rely on the inhibition of eye growth relative to an untreated myopic control group. In fact, such comparisons to untreated myopes lead to reduction rates of 60% of myopic eye growth due to therapy at most, which leaves a apparent “therapeutic gap” of, in this case, 40% or more. But these comparisons are greatly misleading and do not reflect the actual efficiency, and thus, therapeutic value of a therapy option.

In a particular embodiment, the criterion for determining the effectiveness of a current myopia treatment employed thus is the TAG function, which is a combination of a linear equation (y=b+ax) where TAG is a function of subject's age (CA) and declines when age increases, and a constant (y=c) where TAG is independent of subject's age (CA) (TAG criterion). According to this invention the subject's relevant age (CA) for the transition of the linear function of TAG into a constant which is 13.00 yr. It has been surprisingly found that for a very reliable and relevant approach for therapy evaluation and monitoring the TAG criterion according to the invention can be employed in an easy and safe manner to reliably identify insufficient myopia therapy in a short period of time, preferably within a period of less than 12 months, more preferably less than 9 months, even more preferred less than 8 months or less than 7 months, of therapy. According to the invention, the current therapy option, in case of insufficiency, can thus be adapted and less life time is lost where a more efficient therapy could have been employed to a greater benefit of the subject suffering from progressing myopia.

According to a primary embodiment, TAG [mm/yr] is computed according to a combination of a linear equation (y=b+ax) and a constant (y=c), wherein

$$b [ mm / yr ] - a [ mm / {yr}^{2} ] \times CA [ yr ] = T A G [ mm / yr ] \geq c [ mm / yr ]$$

According to a particular aspect, the subject's relevant age (CA) for the transition of the linear function of TAG into a constant is between 12.5 to 13.0 yr, more particular 13.0 yr. That is, for CA [yr]≤13 yr: TAG [mm/yr]=b [mm/yr]−a [mm/yr²]×CA [yr], wherein b is a constant and a is the linear coefficient; and for CA [yr]>13 yr: TAG [mm/yr]=c [mm/yr], wherein c is a constant

It has been surprisingly found that the transition age between 12.5 and 13 forms a reliable transition in the TAG criterion, for both sexes, male and female. This is in contradiction to the current art and knowledge which finds a further reduction in eye growth in an cohort of children that do not become myopic but stays emmetropic during development from childhood to adulthood (emmetropic cohort). In contrast, the TAG-criterion according to the present invention, as a major principle, takes into account the eye's developmental stage and average axial length of a myopic cohort of teens older than 12 yr and older than 13 yr and the observed susceptibility of this age group to myopia treatment as compared to myopia treatment at an earlier age. There is a general declining efficiency of myopia treatment with age which according to the invention determine a less strict TAG criterion than would the normative axial growth rate of the emmetropic cohort. According to the invention, the treatment susceptibility in combination with the normative axial growth rate of an emmetropic cohort, forms a constant function. In preferred variants, c is from 0.08 to 0.12; more preferred c is 0.10.

A further principle underlying the approach of the present invention resides in the identification of a trend of declining efficiency of therapy at very young ages. Without wishing to be bound by the theory, the reduced efficiency of therapy in children at ages 5 to 7 yr is due to the reduced compliance of children and parents and thus adherence to therapy. In a preferred model of the invention, there is thus a less strict TAG criterion employed for children of ages 7 yr or less. Without wishing to be bound by the theory, there is an general optimum curve for myopia therapy efficiency which peaks around subjects' ages of 9 to 11 yr. According to the invention, in a complete model for the TAG criterion to monitor or evaluate a proper myopia treatment, the normative axial growth rate of the emmetropic cohort, which is the treatment goal for the myopia therapy in the first place is offset with an empirically determined optimum curve for general treatment susceptibility and efficiency dependent of the myopic children's age. Thus, in a preferred variant, b is from 0.38 to 0.42 and a is from −0.022 to −0.024. in a more preferred variant of high practical relevance and reliability b is from 0.395 and a is −0.023, and preferably in a combination where c is 0.10.

In more elaborated, yet preferred embodiments, the normative data of the emmetropic eyes underlying the TAG-modelling is taken from matched cohorts of the same ethnicity. There is clear evidence that myopia progression is mainly related to the socio-economic status and visual environment independent of ethnicity and nationality. We had seen a strong increase in myopia prevalence in East Asia, as these countries developed to modern economic standards and high education standards. We did not see a considerable increase in myopia prevalence in Western countries where socio-economic standards and education systems did not change for more than a century. Asian eyes have general tendency to a shallower anterior segment of the eye which determines a longer axial length even for the status of emmetropia. Without wishing to be bound by the theory, while Asian eyes may be longer, they do not show a considerably higher growth rate as compared to eyes of Caucasians. when raised in the same socio-economic and cultural environment. This is in particular true for cohorts of both, Caucasian and Asian children that do not become myopic but stay emmetropic during development from childhood to adulthood (Caucasian emmetropic cohort, Asian emmetropic cohort). The present invention thus pertains to the application of the same TAG criterion for all ethnicities, which results in a very reliable and robust myopia control management.

In an alternative approach, the present invention applies a more reasonable distinction between a TAG criterion which is based on data exclusively from a Caucasian emmetropic cohort and a TAG criterion which is based on data exclusively from an Asian emmetropic cohort, respectively. Thus, in a preferred variant, b is 0.395, if the subject's ethnicity (E) is “Caucasian”, and b is 0.420 if subject's ethnicity is “East Asian”. For other ethnicities, it is preferred but not required, to generally employ the TAG criterion for E=“Caucasian”.

In more elaborated, yet preferred embodiments, the normative data of the emmetropic eyes underlying the TAG-modelling is taken from matched cohorts with the same sex. It has been found that male and female children and adults differ in general size of the eyes, with emmetropic boys having generally larger eyes, and thus axial length than emmetropic girls. The present invention thus pertains to the application of the same TAG criterion for both sexes, which results in a very reliable and robust myopia control management.

In an alternative approach, the present invention applies a more reasonable distinction between a TAG criterion which is based on data exclusively from a male emmetropic cohort and a TAG criterion which is based on data exclusively from a female emmetropic cohort, respectively. Emmetropic girls have the general tendency to a higher growth rate during development between about 6 and 12 years. Thus, in a preferred variant, a is −0.022, if the subject's sex(S) is “male”, and a is −0.024, if subject's sex is “female”.

In particularly preferred variants and further alternatives, the parameters and coefficients in the TAG criterion of the invention are listed in Table 1 as follows:

TABLE 1

| Variant | | | |
|---|---|---|---|
| No.: | a | b | c |
| 1 | from −0.022 to −0.024 | from 0.38 to 0.42 | from 0.08 to 0.12 |
| 2 | from −0.022 to −0.024 | from 0.38 to 0.42 | from 0.10 to 0.12 |
| 3 | from −0.022 to −0.024 | from 0.38 to 0.42 | from 0.08 to 0.10 |
| 4 | from −0.022 to −0.024 | from 0.38 to 0.42 | 0.10 |
| 5 | from −0.022 to −0.024 | from 0.38 to 0.40 | from 0.08 to 0.12 |
| 6 | from −0.022 to −0.024 | from 0.38 to 0.40 | from 0.10 to 0.12 |
| 7 | from −0.022 to −0.024 | from 0.38 to 0.40 | from 0.08 to 0.10 |
| 8 | from −0.022 to −0.024 | from 0.38 to 0.40 | 0.10 |
| 9 | from −0.022 to −0.024 | from 0.40 to 0.42 | from 0.08 to 0.12 |
| 10 | from −0.022 to −0.024 | from 0.40 to 0.42 | from 0.10 to 0.12 |
| 11 | from −0.022 to −0.024 | from 0.40 to 0.42 | from 0.08 to 0.10 |
| 12 | from −0.022 to −0.024 | from 0.40 to 0.42 | 0.10 |
| 13 | from −0.022 to −0.024 | 0.38 | from 0.08 to 0.12 |
| 14 | from −0.022 to −0.024 | 0.38 | from 0.10 to 0.12 |
| 15 | from −0.022 to −0.024 | 0.38 | from 0.08 to 0.10 |
| 16 | from −0.022 to −0.024 | 0.38 | 0.10 |
| 17 | from −0.022 to −0.024 | 0.39 | from 0.08 to 0.12 |
| 18 | from −0.022 to −0.024 | 0.39 | from 0.10 to 0.12 |
| 19 | from −0.022 to −0.024 | 0.39 | from 0.08 to 0.10 |
| 20 | from −0.022 to −0.024 | 0.39 | 0.10 |
| 21 | from −0.022 to −0.024 | 0.395 | from 0.08 to 0.12 |
| 22 | from −0.022 to −0.024 | 0.395 | from 0.10 to 0.12 |
| 23 | from −0.022 to −0.024 | 0.395 | from 0.08 to 0.10 |
| 24 | from −0.022 to −0.024 | 0.395 | 0.10 |
| 25 | from −0.022 to −0.024 | 0.40 | from 0.08 to 0.12 |
| 26 | from −0.022 to −0.024 | 0.40 | from 0.10 to 0.12 |
| 27 | from −0.022 to −0.024 | 0.40 | from 0.08 to 0.10 |
| 28 | from −0.022 to −0.024 | 0.40 | 0.10 |
| 29 | from −0.022 to −0.024 | 0.41 | from 0.08 to 0.12 |
| 30 | from −0.022 to −0.024 | 0.41 | from 0.10 to 0.12 |
| 31 | from −0.022 to −0.024 | 0.41 | from 0.08 to 0.10 |
| 32 | from −0.022 to −0.024 | 0.41 | 0.10 |
| 33 | from −0.022 to −0.024 | 0.42 | from 0.08 to 0.12 |
| 34 | from −0.022 to −0.024 | 0.42 | from 0.10 to 0.12 |
| 35 | from −0.022 to −0.024 | 0.42 | from 0.08 to 0.10 |
| 36 | from −0.022 to −0.024 | 0.42 | 0.10 |
| 37 | from −0.020 to −0.022 | from 0.38 to 0.42 | from 0.08 to 0.12 |
| 38 | from −0.020 to −0.022 | from 0.38 to 0.42 | from 0.10 to 0.12 |
| 39 | from −0.020 to −0.022 | from 0.38 to 0.42 | from 0.08 to 0.10 |
| 40 | from −0.020 to −0.022 | from 0.38 to 0.42 | 0.10 |
| 41 | from −0.020 to −0.022 | from 0.38 to 0.40 | from 0.08 to 0.12 |
| 42 | from −0.020 to −0.022 | from 0.38 to 0.40 | from 0.10 to 0.12 |
| 43 | from −0.020 to −0.022 | from 0.38 to 0.40 | from 0.08 to 0.10 |
| 44 | from −0.020 to −0.022 | from 0.38 to 0.40 | 0.10 |
| 45 | from −0.020 to −0.022 | from 0.40 to 0.42 | from 0.08 to 0.12 |
| 46 | from −0.020 to −0.022 | from 0.40 to 0.42 | from 0.10 to 0.12 |
| 47 | from −0.020 to −0.022 | from 0.40 to 0.42 | from 0.08 to 0.10 |
| 48 | from −0.020 to −0.022 | from 0.40 to 0.42 | 0.10 |
| 49 | from −0.020 to −0.022 | 0.38 | from 0.08 to 0.12 |
| 50 | from −0.020 to −0.022 | 0.38 | from 0.10 to 0.12 |
| 51 | from −0.020 to −0.022 | 0.38 | from 0.08 to 0.10 |
| 52 | from −0.020 to −0.022 | 0.38 | 0.10 |
| 53 | from −0.020 to −0.022 | 0.39 | from 0.08 to 0.12 |
| 54 | from −0.020 to −0.022 | 0.39 | from 0.10 to 0.12 |
| 55 | from −0.020 to −0.022 | 0.39 | from 0.08 to 0.10 |
| 56 | from −0.020 to −0.022 | 0.39 | 0.10 |
| 57 | from −0.020 to −0.022 | 0.395 | from 0.08 to 0.12 |
| 58 | from −0.020 to −0.022 | 0.395 | from 0.10 to 0.12 |
| 59 | from −0.020 to −0.022 | 0.395 | from 0.08 to 0.10 |
| 60 | from −0.020 to −0.022 | 0.395 | 0.10 |
| 61 | from −0.020 to −0.022 | 0.40 | from 0.08 to 0.12 |
| 62 | from −0.020 to −0.022 | 0.40 | from 0.10 to 0.12 |
| 63 | from −0.020 to −0.022 | 0.40 | from 0.08 to 0.10 |
| 64 | from −0.020 to −0.022 | 0.40 | 0.10 |
| 65 | from −0.020 to −0.022 | 0.41 | from 0.08 to 0.12 |
| 66 | from −0.020 to −0.022 | 0.41 | from 0.10 to 0.12 |
| 67 | from −0.020 to −0.022 | 0.41 | from 0.08 to 0.10 |
| 68 | from −0.020 to −0.022 | 0.41 | 0.10 |
| 69 | from −0.020 to −0.022 | 0.42 | from 0.08 to 0.12 |
| 70 | from −0.020 to −0.022 | 0.42 | from 0.10 to 0.12 |
| 71 | from −0.020 to −0.022 | 0.42 | from 0.08 to 0.10 |
| 72 | from −0.020 to −0.022 | 0.42 | 0.10 |
| 73 | from −0.022 to −0.024 | from 0.38 to 0.42 | from 0.08 to 0.12 |
| 74 | from −0.020 to −0.024 | from 0.38 to 0.42 | from 0.10 to 0.12 |
| 75 | from −0.022 to −0.024 | from 0.38 to 0.42 | from 0.08 to 0.10 |
| 76 | from −0.020 to −0.024 | from 0.38 to 0.42 | 0.10 |
| 77 | from −0.022 to −0.024 | from 0.38 to 0.40 | from 0.08 to 0.12 |
| 78 | from −0.020 to −0.024 | from 0.38 to 0.40 | from 0.10 to 0.12 |
| 79 | from −0.022 to −0.024 | from 0.38 to 0.40 | from 0.08 to 0.10 |
| 80 | from −0.020 to −0.024 | from 0.38 to 0.40 | 0.10 |
| 81 | from −0.022 to −0.024 | from 0.40 to 0.42 | from 0.08 to 0.12 |
| 82 | from −0.020 to −0.024 | from 0.40 to 0.42 | from 0.10 to 0.12 |
| 83 | from −0.022 to −0.024 | from 0.40 to 0.42 | from 0.08 to 0.10 |
| 84 | from −0.020 to −0.024 | from 0.40 to 0.42 | 0.10 |
| 85 | from −0.022 to −0.024 | 0.38 | from 0.08 to 0.12 |
| 86 | from −0.020 to −0.024 | 0.38 | from 0.10 to 0.12 |
| 87 | from −0.022 to −0.024 | 0.38 | from 0.08 to 0.10 |
| 88 | from −0.020 to −0.024 | 0.38 | 0.10 |
| 89 | from −0.022 to −0.024 | 0.39 | from 0.08 to 0.12 |
| 90 | from −0.020 to −0.024 | 0.39 | from 0.10 to 0.12 |
| 91 | from −0.022 to −0.024 | 0.39 | from 0.08 to 0.10 |
| 92 | from −0.020 to −0.024 | 0.39 | 0.10 |
| 93 | from −0.022 to −0.024 | 0.395 | from 0.08 to 0.12 |
| 94 | from −0.020 to −0.024 | 0.395 | from 0.10 to 0.12 |
| 95 | from −0.022 to −0.024 | 0.395 | from 0.08 to 0.10 |
| 96 | from −0.020 to −0.024 | 0.395 | 0.10 |
| 97 | from −0.022 to −0.024 | 0.40 | from 0.08 to 0.12 |
| 98 | from −0.020 to −0.024 | 0.40 | from 0.10 to 0.12 |
| 99 | from −0.022 to −0.024 | 0.40 | from 0.08 to 0.10 |
| 100 | from −0.020 to −0.024 | 0.40 | 0.10 |
| 101 | from −0.022 to −0.024 | 0.41 | from 0.08 to 0.12 |
| 102 | from −0.020 to −0.024 | 0.41 | from 0.10 to 0.12 |
| 103 | from −0.022 to −0.024 | 0.41 | from 0.08 to 0.10 |
| 104 | from −0.020 to −0.024 | 0.41 | 0.10 |
| 105 | from −0.022 to −0.024 | 0.42 | from 0.08 to 0.12 |
| 106 | from −0.020 to −0.024 | 0.42 | from 0.10 to 0.12 |
| 107 | from −0.022 to −0.024 | 0.42 | from 0.08 to 0.10 |
| 108 | from −0.020 to −0.024 | 0.42 | 0.10 |
| 109 | from −0.022 to −0.024 | from 0.38 to 0.42 | from 0.08 to 0.12 |
| 110 | from −0.020 to −0.024 | from 0.38 to 0.42 | from 0.10 to 0.12 |
| 111 | from −0.022 to −0.024 | from 0.38 to 0.42 | from 0.08 to 0.10 |
| 112 | from −0.020 to −0.024 | from 0.38 to 0.42 | 0.10 |
| 113 | from −0.022 to −0.024 | from 0.38 to 0.40 | from 0.08 to 0.12 |
| 114 | from −0.020 to −0.024 | from 0.38 to 0.40 | from 0.10 to 0.12 |
| 115 | from −0.022 to −0.024 | from 0.38 to 0.40 | from 0.08 to 0.10 |
| 116 | from −0.020 to −0.024 | from 0.38 to 0.40 | 0.10 |
| 117 | from −0.022 to −0.024 | from 0.40 to 0.42 | from 0.08 to 0.12 |
| 118 | from −0.020 to −0.024 | from 0.40 to 0.42 | from 0.10 to 0.12 |
| 119 | from −0.022 to −0.024 | from 0.40 to 0.42 | from 0.08 to 0.10 |
| 120 | from −0.020 to −0.024 | from 0.40 to 0.42 | 0.10 |
| 121 | from −0.022 to −0.024 | 0.38 | from 0.08 to 0.12 |
| 122 | from −0.020 to −0.024 | 0.38 | from 0.10 to 0.12 |
| 123 | from −0.022 to −0.024 | 0.38 | from 0.08 to 0.10 |
| 124 | from −0.020 to −0.024 | 0.38 | 0.10 |
| 125 | from −0.022 to −0.024 | 0.39 | from 0.08 to 0.12 |
| 126 | from −0.020 to −0.024 | 0.39 | from 0.10 to 0.12 |
| 127 | from −0.022 to −0.024 | 0.39 | from 0.08 to 0.10 |
| 128 | from −0.020 to −0.024 | 0.39 | 0.10 |
| 129 | from −0.022 to −0.024 | 0.395 | from 0.08 to 0.12 |
| 130 | from −0.020 to −0.024 | 0.395 | from 0.10 to 0.12 |
| 131 | from −0.022 to −0.024 | 0.395 | from 0.08 to 0.10 |
| 132 | from −0.020 to −0.024 | 0.395 | 0.10 |
| 133 | from −0.022 to −0.024 | 0.40 | from 0.08 to 0.12 |
| 134 | from −0.020 to −0.024 | 0.40 | from 0.10 to 0.12 |
| 135 | from −0.022 to −0.024 | 0.40 | from 0.08 to 0.10 |
| 136 | from −0.020 to −0.024 | 0.40 | 0.10 |
| 137 | from −0.022 to −0.024 | 0.41 | from 0.08 to 0.12 |
| 138 | from −0.020 to −0.024 | 0.41 | from 0.10 to 0.12 |
| 139 | from −0.022 to −0.024 | 0.41 | from 0.08 to 0.10 |
| 140 | from −0.020 to −0.024 | 0.41 | 0.10 |
| 141 | from −0.022 to −0.024 | 0.42 | from 0.08 to 0.12 |
| 142 | from −0.020 to −0.024 | 0.42 | from 0.10 to 0.12 |
| 143 | from −0.022 to −0.024 | 0.42 | from 0.08 to 0.10 |
| 144 | from −0.020 to −0.024 | 0.42 | 0.10 |
| 145 | −0.023 | from 0.38 to 0.42 | from 0.08 to 0.12 |
| 146 | −0.023 | from 0.38 to 0.42 | from 0.10 to 0.12 |
| 147 | −0.023 | from 0.38 to 0.42 | from 0.08 to 0.10 |
| 148 | −0.023 | from 0.38 to 0.42 | 0.10 |
| 149 | −0.023 | from 0.38 to 0.40 | from 0.08 to 0.12 |
| 150 | −0.023 | from 0.38 to 0.40 | from 0.10 to 0.12 |
| 151 | −0.023 | from 0.38 to 0.40 | from 0.08 to 0.10 |
| 152 | −0.023 | from 0.38 to 0.40 | 0.10 |
| 153 | −0.023 | from 0.40 to 0.42 | from 0.08 to 0.12 |
| 154 | −0.023 | from 0.40 to 0.42 | from 0.10 to 0.12 |
| 155 | −0.023 | from 0.40 to 0.42 | from 0.08 to 0.10 |
| 156 | −0.023 | from 0.40 to 0.42 | 0.10 |
| 157 | −0.023 | 0.38 | from 0.08 to 0.12 |
| 158 | −0.023 | 0.38 | from 0.10 to 0.12 |
| 159 | −0.023 | 0.38 | from 0.08 to 0.10 |
| 160 | −0.023 | 0.38 | 0.10 |
| 161 | −0.023 | 0.39 | from 0.08 to 0.12 |
| 162 | −0.023 | 0.39 | from 0.10 to 0.12 |
| 163 | −0.023 | 0.39 | from 0.08 to 0.10 |
| 164 | −0.023 | 0.39 | 0.10 |
| 165 | −0.023 | 0.395 | from 0.08 to 0.12 |
| 166 | −0.023 | 0.395 | from 0.10 to 0.12 |
| 167 | −0.023 | 0.395 | from 0.08 to 0.10 |
| 168 | −0.023 | 0.395 | 0.10 |
| 169 | −0.023 | 0.40 | from 0.08 to 0.12 |
| 170 | −0.023 | 0.40 | from 0.10 to 0.12 |
| 171 | −0.023 | 0.40 | from 0.08 to 0.10 |
| 172 | −0.023 | 0.40 | 0.10 |
| 173 | −0.023 | 0.41 | from 0.08 to 0.12 |
| 174 | −0.023 | 0.41 | from 0.10 to 0.12 |
| 175 | −0.023 | 0.41 | from 0.08 to 0.10 |
| 176 | −0.023 | 0.41 | 0.10 |
| 177 | −0.023 | 0.42 | from 0.08 to 0.12 |
| 178 | −0.023 | 0.42 | from 0.10 to 0.12 |
| 179 | −0.023 | 0.42 | from 0.08 to 0.10 |
| 180 | −0.023 | 0.42 | 0.10 |
| 181 | −0.023 | from 0.38 to 0.42 | from 0.08 to 0.12 |
| 182 | −0.023 | from 0.38 to 0.42 | from 0.10 to 0.12 |
| 183 | −0.023 | from 0.38 to 0.42 | from 0.08 to 0.10 |
| 184 | −0.023 | from 0.38 to 0.42 | 0.10 |
| 185 | −0.023 | from 0.38 to 0.40 | from 0.08 to 0.12 |
| 186 | −0.023 | from 0.38 to 0.40 | from 0.10 to 0.12 |
| 187 | −0.023 | from 0.38 to 0.40 | from 0.08 to 0.10 |
| 188 | −0.023 | from 0.38 to 0.40 | 0.10 |
| 189 | −0.023 | from 0.40 to 0.42 | from 0.08 to 0.12 |
| 190 | −0.023 | from 0.40 to 0.42 | from 0.10 to 0.12 |
| 191 | −0.023 | from 0.40 to 0.42 | from 0.08 to 0.10 |
| 192 | −0.023 | from 0.40 to 0.42 | 0.10 |
| 193 | −0.023 | 0.38 | from 0.08 to 0.12 |
| 194 | −0.023 | 0.38 | from 0.10 to 0.12 |
| 195 | −0.023 | 0.38 | from 0.08 to 0.10 |
| 196 | −0.023 | 0.38 | 0.10 |
| 197 | −0.023 | 0.39 | from 0.08 to 0.12 |
| 198 | −0.023 | 0.39 | from 0.10 to 0.12 |
| 199 | −0.023 | 0.39 | from 0.08 to 0.10 |
| 200 | −0.023 | 0.39 | 0.10 |
| 201 | −0.023 | 0.395 | from 0.08 to 0.12 |
| 202 | −0.023 | 0.395 | from 0.10 to 0.12 |
| 203 | −0.023 | 0.395 | from 0.08 to 0.10 |
| 204 | −0.023 | 0.395 | 0.10 |
| 205 | −0.023 | 0.40 | from 0.08 to 0.12 |
| 206 | −0.023 | 0.40 | from 0.10 to 0.12 |
| 207 | −0.023 | 0.40 | from 0.08 to 0.10 |
| 208 | −0.023 | 0.40 | 0.10 |
| 209 | −0.023 | 0.41 | from 0.08 to 0.12 |
| 210 | −0.023 | 0.41 | from 0.10 to 0.12 |
| 211 | −0.023 | 0.41 | from 0.08 to 0.10 |
| 212 | −0.023 | 0.41 | 0.10 |
| 213 | −0.023 | 0.42 | from 0.08 to 0.12 |
| 214 | −0.023 | 0.42 | from 0.10 to 0.12 |
| 215 | −0.023 | 0.42 | from 0.08 to 0.10 |
| 216 | −0.023 | 0.42 | 0.10 |

end of Table 1

Even more preferred variants (Table 1) are No. 208, No. 204, No. 164, No. 172, and No. 200.

The disclosure relates to a TAG criterion which follows, with a margin, the average annual growth rates of eyes of children that remain become emmetropic as adolescents and young adults (emmetropic cohort). It is contemplated that the average growth rates of such emmetropic cohorts may be adapted over time as more epidemiological data will be available, specifically for emmetropic cohorts of different ethnicity. The average width of a margin may be selected on the basis of the expected general effectiveness of the myopia treatment option to be monitored and the accuracy and repeatability of the biometric measurements. Currently, a wider margin is preferred to ensure a safe and reliable indication and treatment monitoring and thus an overall more efficient myopia treatment for the subject. It is contemplated, without wishing to be bound by the theory, that a too narrow margin or a too ambitious TAG criterion will likely result in an “overshoot” in the myopia control by to a too fast and/or too extreme change in the treatment option by the eye care provider, such as an excessive dose escalation in the case of pharmacological intervention or too quick switches to new or additional treatment option. This is considered to impair patient's compliance and thus adherence to the myopia therapy, thus eventually resulting in an overall less efficient therapy.

As myopia treatment options will improve in the future, or as the most efficient treatment options known to date, such as multifocal contact lenses or multiple defocus incorporated singe vision glasses (e.g. D.I.M.S.), will become the new standard treatment option and/or as biometric devices apt for myopia control management will improve in handling, operability for a more reliable use in children thus yielding higher accuracy and in particular higher repeatability of the measurements, the smaller the margin the may be selected in a more ambitious approach in myopia control management. The invention thus also pertains to a TAG criterion which approximates the growth rate of an age-matched emmetropic cohort, with a margin of 25% above that ideal, with a margin of 20% above that ideal, with a margin of 15% above that ideal, with a margin of 12% above that ideal, or with a margin of 10% above that ideal. According to further embodiments, TAG [mm/yr] is computed as the age matched annual axial growth rate of a emmetropic cohort [mm/yr] plus a margin of one of: 25%, 20%, 15%, 12%, and 10%.

According to a preferred variant of the invention, the subject's current age (CA) is between 6.00 and 16.50. Without wishing to be bound by the theory, children younger than 6 yr, that is younger than children that attend school and do reading and homework on a regular basis, may exhibit a myopia which may not be fully related to an excessive axial growth (axial myopia) triggered by environmental factors such as lack of time spending outdoors and excessive reading and near work, and may thus not be responsive to current treatment options to reduce or prevent myopia progression by reducing axial growth. In fact, many children under the age of 6 already diagnosed with myopia suffer from a more severe, yet a more genetically than environmentally determined myopia which is less or not susceptible to myopia control management. Rather, for the sake of being practically useful, reliable and sustainable, the current model and method limits itself to the great majority of children who develop myopia only during their school ages and it does not embrace further detailed models for outliers which show high myopia already at very young ages.

According to a preferred variant of the invention, the subject's eye's degree of myopia, at the time of the start of the therapy using the method of the invention, referred to as the spherical equivalent (SER), is from 0.0 Dpt. (Diopters) to −8.0 Dpt.; in more preferred variants, in the order of increasing preference, the degree of myopia is from 0.0 to −7.0 Dpt., from 0.0 to −6.0 Dpt., from 0.0 to −5.0 Dpt., from 0.0 to −4.0 Dpt., from 0.0 to −3.0 Dpt., from 0.0 to −2.0 Dpt., from 0.0 to −1.5 Dpt.; in other preferred variants, in the order of increasing preference, from −0.5 to −7.0 Dpt., from −0.5 to −6.0 Dpt., from −0.5 to −5.0 Dpt., from −0.5 to −4.0 Dpt., from −0.5 to −3.0 Dpt., from −0.5 to −2.0 Dpt., from −0.5 to −1.5 Dpt.; in other preferred variants, in the order of increasing preference, from −1.0 to −7.0 Dpt., from −1.0 to −6.0 Dpt., from −1.0 to −5.0 Dpt., from −1.0 to −4.0 Dpt., from −1.0 to −3.0 Dpt., from −1.0 to −2.0 Dpt.; in other preferred variants, in the order of increasing preference, from −1.5 to −7.0 Dpt., from −1.5 to −6.0 Dpt., from −1.5 to −5.0 Dpt., from −1.5 to −4.0 Dpt., from −1.5 to −3.5 Dpt.; in other preferred variants, in the order of increasing preference, from −2.0 to −7.0 Dpt., from −2.0 to −6.0 Dpt., from −2.0 to −5.0 Dpt., from −2.0 to −4.0 Dpt., from −2.0 to −3.5 Dpt.

In preferred variants of the invention, the subject's eye's rate of annualized myopic progression, present at the time of the start of the therapy using the method of the invention, is from −0.25 Dpt./yr (Diopters per year) to −2.5 Dpt./yr; in more preferred variants, in the order of increasing preference, the rate of annualized myopic progression is from −0.25 Dpt./yr to −2.0 Dpt./yr, from −0.25 Dpt./yr to −1.5 Dpt./yr, or from −0.25 Dpt./yr to −1.0 Dpt./yr; in other preferred variants, in the order of increasing preference, the rate of annualized myopic progression is from −0.05 Dpt./yr to −2.5 Dpt./yr, from −0.50 Dpt./yr to −2.0 Dpt./yr, from −0.50 Dpt./yr to −1.5 Dpt./yr or from −0.50 Dpt./yr to −1.0 Dpt./yr.

In more preferred variants of the invention, the subject's eye's rate of myopic progression is assessed by the axial growth rate, which in accordance with the idea of the present invention can be assessed more easily and reliably by means of a modern up-to-date biometric device. Accordingly it is preferred that the subject's eye's axial growth rate (AR) present at the time of the start of the therapy using the method of the invention, is from 0.55 mm/yr (Millimeters per year) to 0.25 mm/yr in the group of children being younger than 10.0 yr, and from 0.35 mm/yr to 0.14 mm/yr in the group of children being 10.0 yr or older. In more preferred variants in the group of children being younger than 10.0 yr, the axial growth rate is in the order of increasing preference from 0.50 mm/yr to 0.25 mm/yr, 0.45 mm/yr to 0.25 mm/yr, 0.40 mm/yr to 0.25 mm/yr, or 0.35 mm/yr to 0.25 mm/yr. In more preferred variants in the group of children being 10.0 yr of age or older, the axial growth rate is from 0.30 mm/yr to 0.14 mm/yr, 0.28 mm/yr to 0.14 mm/yr, 0.26 mm/yr to 0.14 mm/yr, 0.24 mm/yr to 0.14 mm/yr, 0.22 mm/yr to 0.14 mm/yr, 0.20 mm/yr to 0.14 mm/yr, or 0.18 mm/yr to 0.14 mm/yr; in other preferred variants, from 0.30 mm/yr to 0.18 mm/yr, 0.28 mm/yr to 0.18 mm/yr, 0.26 mm/yr to 0.18 mm/yr, 0.24 mm/yr to 0.18 mm/yr, or 0.22 mm/yr to 0.18 mm/yr; and in other preferred variants, from 0.30 mm/yr to 0.20 mm/yr, 0.28 mm/yr to 0.20 mm/yr, 0.26 mm/yr to 0.20 mm/yr, or 0.24 mm/yr to 0.20 mm/yr, or from 0.26 mm/yr to 0.22 mm/yr.

The invention further pertains to a method for treating myopia a subject in need thereof, in particular a subject being diagnosed with current or incident myopia, in particular progressive myopia, or being at risk of developing the same, in at least one eye (myopia treatment), where within the treatment the efficiency of the myopia treatment is tracked or monitored by the steps (a) to (e) according to the invention. Most preferred, the method includes an indication on if and how to select the appropriate myopia treatment options to which the treated subject is most susceptible to. This may also include considerations on the subject's individual preferences to particular treatment options and expected subject's individual adherence thereto.

The method of treating myopia may further comprises the step (f) where in case of step (e) the output comprises the information that the myopia treatment is currently insufficient or ineffective, a further treatment measure is taken. It is contemplated that the further or alternative treatment option intensifies the myopia treatment and/or increases the subject's individual response or susceptibility to myopia treatment.

Furthermore, the invention pertains to a method for treating a subject, the subject suffering from or being at risk of developing a condition which is a sight-threatening disease, which is related to or being, at least in part, caused by myopia or high myopia. The condition is selected from: Glaucoma, in particular open-angle glaucoma, cataract, in particular nuclear, cortical, and posterior subcapsular cataract, peripapillary deformation, posterior staphyloma, dome-shaped macula, choroidal/scleral thinning, myopic choroidal neovascularization retinal tears, retinal detachment, myopic maculopathy, and myopic macular degeneration. This method comprising tracking or monitoring the efficiency of the myopia treatment by a method comprising the steps (a) to (e) and optionally (f).

In the context of the present invention, the terms “treatment” or “treating” are used herein to denote delaying the onset of, preventing, inhibiting, alleviating the effects of, or regressing a disease or a symptom thereof, or any side-effect related thereto, in a subject.

In the context of the present invention, the term “high myopia” is used to describe a condition of myopia which, according to the definition of the World Health Organization, is reflected in a spherical equivalent of −5 Dpt. or higher. In an alternative setting, the term “high myopia” describes a condition of myopia of a spherical equivalent of −6 Dpt. or higher.

In the context of the present invention, the term “subject” is used to describe an human or non-human individual, to whom treatment according to the methods of the present disclosure is provided. Human applications are anticipated by the present disclosure. Both, pediatric and adult subjects are included. In any of the methods described herein, the subject can be at least 18 months old, e.g., 18 months or older, 2 years or older, 4 years or older, 6 years or older, 8 years or older, 10 years or older, 12 years or older, 13 years or older, 14 years or older, 16 years or older, 18 years or older, 21 years or older, 25 years or older, or 30 years or older. In any of the methods described herein, the subject can at the same time be 5 years or younger, 7 years or younger, 9 years or younger, 11 years or younger, 13 years or younger, 15 years or younger, 18 years or younger, 21 years or younger, 25 years or younger, or 30 years or younger.

The invention further pertains to a computer-based system for automated evaluation and monitoring of myopia treatment efficiency, the monitoring system comprises at least: a computing device, a computer memory for storing program instructions, and a CPU for operating the stored program instructions, said program instructions comprising the instruction steps according to the invention. In a variant, the system is in the form of a remotely operable application in a network environment or cloud setup which connects to a biometry device to receive biometry data, i.e. axial length data, or receives the biometry data via direct data input on a data input device, such as a keyboard. The system may further comprise a database in data communication with the CPU and/or memory, for recording biometry data, i.e. in particular, but not limited to current axial length (CAX) of a subject's eye in connection with the subject's current age (CA) or the current date and subject's date of birth, respectively, and for retrieving one or more past recorded axial lengths (PAX) and the subject's previous ages (PA) or date of recording, respectively.

The setup preferably comprises as biometry device (21) for automated measurement of the axial length of a subject's eye which is in data connection via a data interface (22) with a computing device (23). The biometry device may be self-contained and operable in itself to perform the measurement autonomously and the interface transfers the biometry data, during or sometime after the measurement, to the computing device (23) for computing and processing. The data interface (22) in its simplest form may be a data cable for serial communication, but may also comprise a network setup with data server for storage and retrieval of the biometry data. The network may also include one or more cloud based storage and processing steps. In a preferred variant, the connection of the biometry device (21) to the computing device (23) also includes a control connection for remotely controlling data acquisition or mode changes in the biometry device (21) via the instructions running on the computing device (23). In a preferred variant, the computing device (23), and preferably all its components, is located directly within the biometry device (21). Examples of current biometry devices of measuring a subject's axial length employ optical biometry, and include coherence interferometry, optical low-coherence reflectometry techniques, but also include imaging techniques apt to assess axial length, such as swept-source optical coherence tomography. Preferred current optical biometry devices include, but are not limited to Lenstar LS900 (Haag-Streit Diagnostics, Switzerland), MyopiaMaster and Pentacam AXL (Oculus, Germany), IOLMaster 500 and IOLMaster 700 (Carl Zeiss, Germany), OA-2000 (Tomey, Japan), and Aladdin HW and MYAH (Topcon, The Netherlands).

The computing device (23) includes, at least one database (24) which may be enclosed within the computing device (23) or may be remote database in a network setup with a data server for storage and retrieval of the data. The database (24) network may also include one or more cloud based storage and processing steps. The database is mainly for storing and recalling subject's data, including, but not limited to visit/measurement dates, date of birth, biometry data of all past and current measurements.

The computing device (23) also includes, at least one processing unit CPU (25), configured to execute instructions, which is preferably enclosed within the computing device (23) or may be a remote processing unit or process running in a network environment. It is contemplated that the CPU is in the form of a dedicated hardware processor or a virtual processing unit running in a virtual environment (virtual machine). In data and control connection to the CPU (25) there is a memory (26) for storing the computing results and/or the program instructions. It is contemplated that the memory (26) is in the form of random access memory (RAM) and/or cache memory or read-only memory (ROM), such as, e.g. memory space for storing program instructions on a hardware CPU chip, but also includes other removable or non-removable, volatile or non-volatile storage media known as such.

The computing device (23), in a preferred variant, also includes, a network device (28), which preferably is enclosed with the computing device (23) or is in data connection with the computing device (23), for connecting the computing device (23) to a network environment, including remote access to the computing device (23). In is contemplated that the network device (28) may utilize all useful wireless protocols including Bluetooth, WIFI (e.g., 802.11a/b/g/n), cellular network (CDMA, GSM, M2M, 3G/4G/LTE, and 5G). As described, the network environment may include, but is not limited to database (24), data input device (27), data display device (28), and biometry device (21).

The system further includes, at least one data input device (27) which may be enclosed within the computing device (23) or is in data connection with the computing device (23) and, in an alternative variant, is a remote data input terminal in a network environment including a data display and input application running on a mobile handheld device. The data input device (27) is mainly for user input of data, in particular, but not limited to, a patient's biometric data and date of birth. The data input device (27) primarily is a human interface and is in a particular variant a physical keyboard, but also encompasses touch screen keyboards and physical pointing devices and touch screen devices as well as virtual input devices in a VR environment. In a preferred variant the input device (27) is in the form of a touch screen for data input located directly on the user operation interface of the biometry device (21).

The system further includes, at least one data display device (28) which may be enclosed within the computing device (23) or is in data connection with the computing device (23) and, in an alternative variant, is a remote data display terminal in a network environment, including a display application running on a mobile handheld device. The data display device (28) is mainly for user display of data, in particular, but not limited to, the result of the computing instructions according to the invention, a patient's biometric data and personal information. The data display device (28) primarily is a human interface and is in a particular a display screen such as a computer monitor, but also virtual display devices in a VR environment. In a preferred variant, the display device (27) is in the form of a display screen located directly on the user operation surface of the biometry device (21).

The invention also pertains to a computer program product which comprises or consists of a non-volatile readable storage medium or a data server, which have embodied the program instructions according to the method of invention. In a variant, the computer program product is a physical memory stick for data storage or data space on a physical server or in a cloud computing environment.

### EXAMPLES

The following examples illustrate some of the experiments leading to the models and methods according to the invention, however, should not be construed to limit the scope of the claims in any way.

### Example 1: Target Axial Growth Rate for Myopia Therapy as a Linear Function of Age

A normative curve representing the target axial growth rates for evaluation and monitoring myopia progression and controlling therapy is established taking into account longitudinal and cross-sectional data of European low myopic and emmetropic children of ages 3 to 18 years (n=457, 45% male). All children and their parents consented to the recording, processing and publication of their biometry data in anonymized form. The rules for good clinical practice (GCP-E6) and the tenets of the Declaration of Helsinki (in the latest revision) were followed.

From ages 5 to 16 for both, girls and boys, the model reveals a target axial growth rate that drops constantly with age. Of note, above the age of 13, our model describes a constant target growth rate for both, girls and boys.

FIG. 1 shows the modelled axial growth rates dependent on subject's age that serve as the criteria for therapeutic efficiency in the method according to the invention. The lower curve “IAG” represents the ideal axial growth rates mainly corresponding to a full halt of any significant myopic progression in the treated eye. The upper curve “TAG” represents still tolerable axial growth rates of a treated myopic eye corresponding to a fully sufficient therapy of myopic progression. The dotted fine lines resemble the collection of our clinical data set underlying the TAG and IAG models, respectively.

If the individual growth rate of a subject remains below the TAG line at any time of the treatment, the myopia therapy will be considered highly efficient. If the individual growth rate of a subject stays or returns to above the TAG line during the treatment, the myopia therapy options or measures will be considered not efficient and a change in the therapy option is indicated.

Table 2 shows the functions that represent the Ideal Axial Growth Rate (IAG) as expressed in mm (Millimeters) axial growth per year [mm/yr] dependent on the individual age of the child with a myopic progressing eye. Ideally, a therapy aiming to a full halt of the eye's myopia progression will eventually result in the treated eye growing at IAG, if the therapy was fully effective.

TABLE 2
Ideal Axial Growth Rate (IAG) as a function of Age (A)

| Ideal Axial | | |
|---|---|---|
| Growth Rate | Age (A) [yr] | |
| (IAG) [mm/yr] | 3−14 | >14 |
| male | IAG = 0.283 − 0.0161 * A | IAG = 0.050 |
| female | IAG = 0.317 − 0.0179 * A | IAG = 0.045 |
| both sexes | IAG = 0.30 − 0.017 * A | IAG = 0.047 |

Table 3 shows the functions that represent a tolerable axial growth rate (TAG) for a myopic progressing eye. Ideally, a therapy aiming to efficiently reduce myopia progression shall eventually result in the treated eye growing at maximum at TAG.

TABLE 3
Tolerable Axial Growth Rate (TAG) as a function of Age (A)

| Tolerable Axial Growth | |
|---|---|
| Rate (TAG) [mm/yr] | |
| female | 0.42 − 0.024 * A = TAG ≥ 0.1 |
| male | 0.38 − 0.022 * A = TAG ≥ 0.1 |
| both sexes | 0.40 − 0.023 * A = TAG ≥ 0.1 |

According to this exemplary embodiment, the minimum tolerable axial growth rate is 0.1 mm/yr. A comparable growth rate is observed among Caucasian children of the same age, who will have a mean axial length of 23.33 mm (female) and 23.77 mm (male), which is commonly associated with emmetropia, at adulthood (at age of 18 yr).

### Example 2: Device for Automated Monitoring of Myopia Therapy

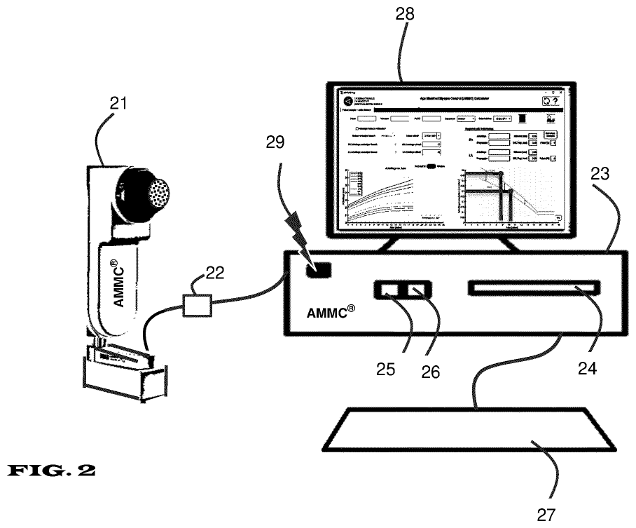

FIG. 2 schematically depicts a preferred embodiment of a system for the automated assessment of therapeutic efficiency of a myopia treatment option. The setup comprises as biometry device (21) for automated measurement of the axial length of a subject's eye which is in data connection via a data interface (22) with a computing device (23). The data interface (22) may comprise a network setup with data server for storage and retrieval of the biometry data. The computing device (23) includes, at least one database (24) which may be enclosed within the computing device (23) or may be remote database in a network setup with a data server for storage and retrieval of the data. The computing device (23) also includes, at least one processing unit CPU (25). In data and control connection to the CPU (25) there is a memory (26) for storing the computing results and/or the program instructions. The computing device (23) may also include a network device (28) for connecting the computing device (23) to a network environment, which may include, but is not limited to database (24), data input device (27), data display device (28), and biometry device (21).

The system further includes, at least one data input device (27) which is in data connection with the computing device (23) for user input of data, in particular, but not limited to, a patient's biometric data and date of birth. The data input device (27) primarily is a human interface and is in a particular variant a physical keyboard.

The system further includes, at least one data display device (28) which is in data connection with the computing device (23) for user display of data, in particular, but not limited to, the result of the computing instructions according to the invention, a patient's biometric data and personal information. The data display device (28) primarily is a human interface and is in a particular a display screen such as a computer monitor, In a preferred variant, the display device (27) is in the form of a display screen located directly on the user operation surface of the biometry device (21).

### Example 3: Monitoring of Myopia Therapy in a Clinical Environment

In a controlled clinical environment, children of ages 5 to 15 yr (n=122, 56% male) all diagnosed with progressive myopia, were subject to myopia control treatment and were followed over periods of 3 to 5 years. All children and their parents consented to the recording, processing and publication of their biometry data in anonymized form. The rules for good clinical practice (GCP-E6) and the tenets of the Declaration of Helsinki (in the latest revision) were followed.

The myopia control treatment employed included the regular assessment of biometric parameters of the young patients' eyes, including axial length at intervals of 11 to 18 months. All biometry was performed by IOLMaster500 or IOLMaster700 (Carl Zeiss Meditec, Germany). In addition, objective refraction and/or subjective refraction was recorded. In n=88 cases the initial myopia therapy option was low-dose atropine (0.01% or 0.025%; BergApotheke or Anna Apotheke, Ursensollen, Germany), in n=30 cases the initial therapy option was bifocal contact lenses, n=4 cases followed some other myopia treatment option as initial therapy. As soon as the current axial growth rate (AG) could be determined, i.e. when two reliable measurements of axial length at an interval of at least 11 months were available, TAG was computed for patient's actual age (CA) according to:

b [mm/yr] − a [mm/yr²] × CA [yr] = TAG [mm/yr] ≥ c [mm/yr]
with a = −0.023, b = 0.40, and c = 0.1 (see Example 1 and Variant 208).

For all instances where TAG (CA) remained below AG, a new therapeutic strategy was immediately selected. In n=29 cases the myopia therapy option was switched from 0.01% to 0.025% dose atropine. In n=2 cases the myopia therapy option was switched from 0.025% to 0.05% dose atropine. In n=8 cases the therapy option was switched from low-dose atropine (0.01% or 0.025%) to a combination therapy of bifocal contact lenses and low-dose atropine. In n=18 cases the therapy option was switched from bifocal contact lenses to a combination therapy of bifocal contact lenses and low-dose atropine (0.025%). All n=4 cases following other myopia treatment options were switched to 0.025% dose atropine. In n=7 cases the therapy option was switched from low-dose atropine (0.01% or 0.025%) to a combination therapy of DIMS glasses and low-dose atropine. In n=8 cases the therapy option was switched from bifocal contact lenses to a DIMS glasses. Note that for patients that were subject to low-dose atropine therapy or to dose escalation in low-dose atropine therapy, in all cases, except for 3 cases, patients remained with the same atropine formulation (either BergApotheke or Anna Apotheke Ursensollen), due to detected differences in the intensity of the atropine effect between the formulations. In the 3 cases, we switched from the original BergApotheke formulation to the Anna Apotheke formulation as soon as dose escalation was selected as indicated by the method of the invention.

As a result, all subjects followed showed a rest in the progression of myopia or an increase in myopia (myopic shift) which was not more than 1.5 Dpt. for the group followed for 4 years or more (n=32) and not more than 1.0 Dpt. for the group followed for less than 4 years (n=90), respectively.

1. A computer-implemented method for automatic evaluation and monitoring the efficiency of a myopia treatment of a subject's eye, the method comprising the steps of:
(a) obtaining the current age (CA) of the subject;
(b) obtaining the current annualized axial growth rate (AG) of the subject's eye;
(c) calculating a maximum tolerable axial growth rate (TAG) as a function of the subject's current age (CA);
(d) comparing the AG with TAG; and
(e) generating an output on the basis of said comparison, selected from:
(i) output comprising the information of currently insufficient or ineffective myopia therapy for AG greater than TAG, and
(ii) output comprising the information of currently adequate or effective myopia therapy for AG smaller than TAG or equal.

2. The method of claim 1, wherein TAG is computed according to the equation system

| | |
|---|---|
| (eq. 1): | TAG [mm/yr] = b [mm/yr] − a [mm/yr²] × CA [yr]. and |
| (eq. 2): | TAG [mm/yr] ≥ c [mm/yr] |

wherein b is a constant and a is a linear coefficient, and
c is a constant.

3. The method of claim 2, wherein c is from 0.08 to 0.12.

4. The method of claim 2, wherein c is 0.10.

5. The method of claim 2, wherein b is from 0.38 to 0.42 and a is from −0.022 to −0.024.

6. The method of claim 2, wherein b is 0.395, if the subject's ethnicity (E) is “Caucasian”, and b is 0.420 if E is “East Asian”.

7. The method of claim 2, wherein a is −0.022, if the subject's sex(S) is “male”, and a is −0.024, if S is “female”.

8. The method of claim 1, wherein in step (b) comprises the steps of:
(b1) retrieving a past axial length (PAX) [mm] from that eye from a previous point of time and the previous age (PA) [yr] of the subject at that previous point of time from a database record;
(b2) obtaining the current axial length (CAX) from that eye; and
(b3) computing AG according to: AG=(CAX−PAX)/(CA−PA)

9. The method of claim 1, wherein the subject's current age (CA) is between 6.0 and 16.5 yr.

10. The method of claim 1, wherein in step (a) comprises the steps of:
(a1) obtaining the subject's date of birth;
(a2) obtaining the current date; and
(a3) computing the CA from the difference between said dates.

11. The method of claim 1, where the subject's eye's AG is assessed at an interval of 6 months to less than 12 months to monitor the efficiency of the myopia treatment of the subject's eye

12. A computer-based system for the automated evaluation and monitoring of myopia treatment efficiency, comprising in a computing device (23) a computer memory (26) for storing program instructions and a CPU (25) for operating the stored program instructions, said program instructions comprising the steps (a) to (e) of the method according to claim 1.

13. The computer-based system of claim 12 further comprising a database (24), in data communication with said CPU (25) and/or memory (26), for storing current axial length (CAX) of a subject's eye in connection with the subject's current age (CA) and for retrieving one or more past axial lengths (PAX) and the subject's one or more previous ages (PA) connected thereto.

14. The computer-based system of claim 12 further comprising a biometry device (21) for assessing the current axial length (CAX) of a subject's eye.

15. A computer program product comprising a non-volatile readable storage medium or data server having embodied program instructions, said program instructions comprising the steps (a) to (e) of the method according to claim 1.
